(19) United States
(12) Patent Application Publication
(10) Pub. No.: US 2025/0204760 A1
(43) Pub. Date: Jun. 26, 2025

(54) VALVE CAP FOR AN ENDOSCOPE

(71) Applicant: Boston Scientific Scimed, Inc., Maple Grove, MN (US)

(72) Inventor: Rosangel Aimee Ramos Espinoza, Allston, MA (US)

(73) Assignee: Boston Scientific Scimed, Inc., Maple Grove, MN (US)

(21) Appl. No.: 18/987,608

(22) Filed: Dec. 19, 2024

Related U.S. Application Data
(60) Provisional application No. 63/613,567, filed on Dec. 21, 2023.

(51) Int. Cl. A61B 1/015; A61B 1/00

(52) U.S. Cl. CPC A61B 1/015; A61B 1/00137

(57) ABSTRACT

Devices, systems, and methods for a valve assembly for a medical device. The top surface of the valve assembly is widened to accommodate smaller hands and/or fingers. A flange at the valve cap, or a removable extension added to the top of the valve cap, is wider than the internal valve well and allows operation of the valve assembly when fingers can reach the extended portion of the upper surface. Passages connect to air holes where needed.

### CROSS-REFERENCE TO RELATED APPLICATIONS

This application claims the benefit of U.S. Provisional Patent Application Ser. No. 63/613,567 filed on Dec. 21, 2023, the disclosure of which is incorporated herein by reference.

### FIELD

This disclosure relates generally to valve assemblies and methods, and particularly to valve assemblies and methods for an endoscope.

### BACKGROUND

A wide variety of intracorporeal medical devices and systems have been developed for medical use, for example, for endoscopic procedures. Some of these devices and systems include guidewires, catheters, catheter systems, endoscopic instruments, and the like. These devices and systems are manufactured by any one of a variety of different manufacturing methods and may be used according to any one of a variety of methods. Of the known medical devices, systems, and methods, each has certain advantages and disadvantages.

During an endoscopic procedure, an endoscope operator (such as a medical professional) activates valves in order to apply air, CO2, water, and/or suction to one or more working channels of the endoscope. The operator may be expected to press and hold each valve cap using a particular finger, and each valve may be used hundreds of times during a typical procedure.

The placement of the valve caps in endoscopic handles are static, dictated by the size and shape of the device, as well as “default” hand size and shape. However, many operators may have hands and/or fingers that are significantly smaller than the default. If the operator's hand or fingers must be manipulated awkwardly or stretched to reach and press the valve cap, the repetitive motions may become uncomfortable or interfere with efficient operation of the endoscope. There is a need for endoscopic components that can comfortably accommodate a wider range of operators.

### SUMMARY

This disclosure provides design, material, manufacturing method, and use alternatives for medical devices and medical systems. In a first example, a valve assembly for a medical device includes a valve body having an internal valve well; a valve cap positioned within the internal valve well and configured to slide vertically within the internal valve well between an upper and a lower position; a spring member positioned between the valve cap and the valve body such that, when the valve cap is pushed downward relative to the valve body, the spring member applies upward force against the valve cap; and a valve shaft connected to the valve cap and configured to move with the valve cap between an upper and a lower position. The valve cap has a top flange that is wider than the internal valve well.

Alternatively or additionally to any of the examples above, the width of the top flange is more than 20% greater than the width of the internal valve well.

Alternatively or additionally to any of the examples above, the width of the top flange is greater than the width of the valve body.

Alternatively or additionally to any of the examples above, wherein the valve cap has an air escape hole.

Alternatively or additionally to any of the examples above, the valve shaft has an air channel in fluid communication with the air escape hole.

Alternatively or additionally to any of the examples above, wherein the valve assembly is an air/water supply valve.

Alternatively or additionally to any of the examples above, the valve assembly is a suction valve.

In another example, a valve assembly for a medical device includes a valve body having an internal valve well; a valve cap positioned within the internal valve well and configured to slide vertically within the internal valve well between an upper and a lower position; a spring member positioned between the valve cap and the valve body such that, when the valve cap is pushed downward relative to the valve body, the spring member applies upward force against the valve cap; a valve shaft connected to the valve cap and configured to move with the valve cap between an upper and a lower position; and a removable valve cap extension attached to the valve cap, the removable valve cap extension having a protrusion extending past an edge of a top surface of the valve cap.

Alternatively or additionally to any of the examples above, the removable valve cap extension comprises a surface portion configured to attach to the top surface of the valve cap.

Alternatively or additionally to any of the examples above, the surface portion rotationally symmetrical such that the protrusion can be positioned to extend from the top surface of valve cap at different angles.

Alternatively or additionally to any of the examples above, the protrusion of the removable cap extension extends parallel to the top surface of the valve cap.

Alternatively or additionally to any of the examples above, the protrusion of the removable cap extension extends at a downward angle from the surface of the valve cap.

Alternatively or additionally to any of the examples above, the valve cap comprises an air escape hole, and wherein the removable cap extension comprises an air passage in fluid communication with the air escape hole.

Alternatively or additionally to any of the examples above, the air passage of the removable cap extension runs at an angle from a bottom of the surface portion towards a top of the protrusion.

In another example, an endoscopic surgical device includes an operating handle having first and second valve locations; a source of suction in fluid communication with the first valve location; a first valve assembly of any one of the above examples installed in the first valve location; a gas supply in fluid communication with the second valve location; a water supply in fluid communication with the second valve location; and a second valve assembly of any one of the above examples installed in the second valve location.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various embodiments and together with the description serve to explain the principles of the present disclosure.

FIG. 1 depicts a schematic view of components of an illustrative endoscope;

FIG. 2 depicts a schematic view of components of an illustrative endoscope system;

FIG. 3A depicts a perspective view of an illustrative suction valve;

FIG. 3B depicts a perspective view of an illustrative air/water supply valve;

FIG. 4 depicts an operator's hand positioned to manipulate valve caps on an illustrative endoscope handle;

FIG. 5 depicts an operator's hand positioned to manipulate expanded valve caps on an illustrative endoscope handle;

FIG. 6 depicts an operator's hand positioned to manipulate extensions to valve caps on an illustrative endoscope handle;

FIG. 7 depicts an operator's hand positioned to manipulate extensions to valve caps on an illustrative endoscope handle;

FIG. 8A depicts a perspective view of an illustrative suction valve with an expanded cap surface;

FIG. 8B depicts a perspective view of an illustrative air/water supply valve with an expanded cap surface;

FIG. 9A depicts schematic plan and side views of a valve cap extension;

FIG. 9B depicts schematic plan and side views of a valve cap extension;

FIG. 9C depicts a perspective view of an illustrative suction valve with a valve cap extension;

FIG. 9D depicts a perspective view of an illustrative air/water supply valve with a valve cap extension;

FIG. 10A depicts schematic plan and side views of a valve cap extension;

FIG. 10B depicts schematic plan and side views of a valve cap extension;

FIG. 10C depicts a perspective view of an illustrative air/water supply valve with a valve cap extension;

FIG. 11A depicts a side view of an illustrative suction valve with a valve cap extension;

FIG. 11B depicts a side view of an illustrative air/water supply valve with a valve cap extension;

FIG. 11C depicts a side view of an illustrative suction valve with a valve cap extension; and

FIG. 11D depicts a side view of an illustrative air/water supply valve with a valve cap extension.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

This disclosure is now described with reference to an illustrative medical system that may be used in endoscopic medical procedures. However, it should be noted that reference to this particular procedure is provided only for convenience and not intended to limit the disclosure. A person of ordinary skill in the art would recognize that the concepts underlying the disclosed devices and related methods of use may be utilized in any suitable procedure, medical or otherwise. This disclosure may be understood with reference to the following description and the appended drawings, wherein like elements are referred to with the same reference numerals.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Those skilled in the art will recognize that the various elements described may be arranged in various combinations and configurations without departing from the scope of the disclosure. The detailed description illustrates example embodiments of the disclosure.

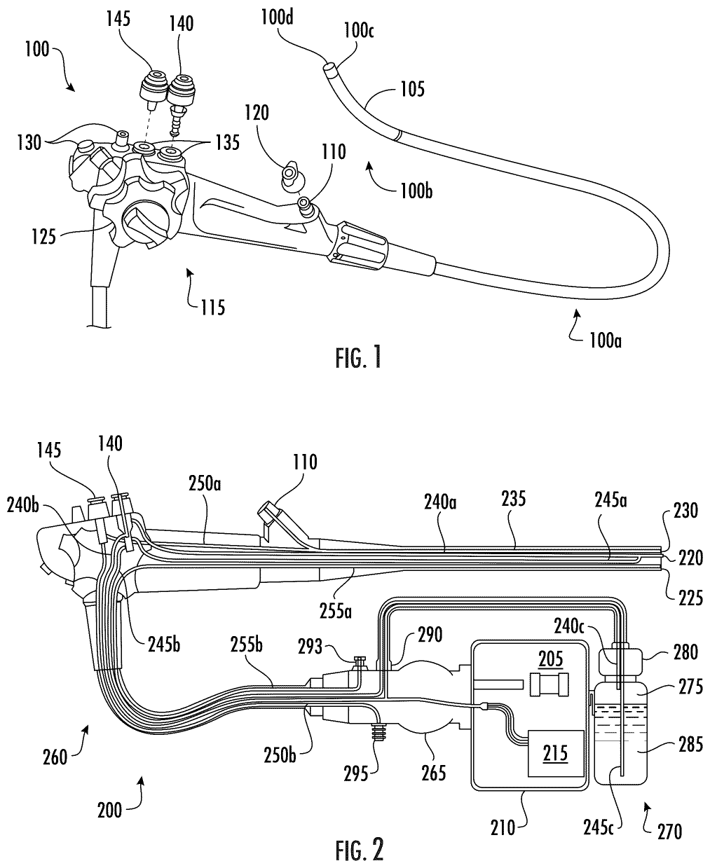

With reference to FIG. 1, an illustrative endoscope 100 is depicted and FIG. 2 depicts an illustrative endoscope system 200. The endoscope 100 may include an elongated tube or shaft 100a that is configured to be inserted into a subject (e.g., a patient).

A light source 205 of the endoscope system 200 may feed illumination light to a distal portion 100b of the endoscope 100. The distal portion 100b of the endoscope 100 may house an imager (e.g., CCD or CMOS imager) (not shown). The light source 205 (e.g., lamp) may be located in a video processing unit 210 that processes signals input from the imager and outputs processed video signals to a video monitor (not shown) for viewing. The video processing unit 210 may also serves as a component of an air/water feed circuit by housing a pressurizing pump 215, such as an air feed pump, in the unit 210.

The endoscope shaft 100a may include a distal tip 100c (e.g., a distal tip unit) provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 220 for supplying gas to insufflate the interior of the patient at the treatment area and for supplying water to wash a lens covering the imager. An irrigation opening 225 in the end face 100d supplies irrigation fluid to the treatment area of the patient. Illumination windows (not shown) that convey illumination light to the treatment area, and an opening 230 to a working channel 235 extending along the shaft 100a for passing tools to the treatment area, may also be included on the face 100d of the distal tip 100c. The working channel 235 may extend along the shaft 100a to a proximal channel opening 110 positioned distal to an operating handle 115 (e.g., a proximal handle) of the endoscope 100. A biopsy valve 120 may be utilized to seal the channel opening 110 against unwanted fluid egress.

The operating handle 115 may be provided with knobs 125 for providing remote 4-way steering of the distal tip via wires connected to the articulation joint in the bendable flexible portion 105 (e.g., one knob controls up-down steering and another knob control for left-right steering). A plurality of video switches 130 for remotely operating the video processing unit 210 may be arranged on a proximal end side of the handle 115.

The handle 115 may be provided with dual valve locations 135. One of the valve locations 135 may receive a gas/water valve 140 for operating an insufflating gas and lens water feed operation. A gas supply line 240a and a lens wash supply line 245a run distally from the gas/water valve 140 along the shaft 100a and converge at the distal tip 100c proximal to the gas/wash nozzle 220 (FIG. 2).

The other valve location 135 may receive a suction valve 145 for operating a suction operation. A suction supply line 250a may run distally from the suction valve 145 along the shaft 100a to a junction point in fluid communication with the working channel 235 of the endoscope 100.

The operating handle 115 may be electrically and fluidly connected to the video processing unit 210, via a flexible umbilical 260 and connector portion 265 extending therebetween. The flexible umbilical 260 has a gas (e.g., air or CO₂) feed line 240b, a lens wash feed line 245b, a suction feed line 250b, an irrigation feed line 255b, a light guide (not shown), and an electrical signal cable (not shown). The connector portion 265 when plugged into the video processing unit 210 connects the light source 205 in the video processing unit with the light guide. The light guide runs along the umbilical 260 and the length of the endoscope shaft 100a to transmit light to the distal tip 100c of the endoscope 100. The connector portion 265 when plugged into the video processing unit 210 also connects the air pump 215 to the gas feed line 240b in the umbilical 260.

A water reservoir or container 270 (e.g., water bottle) may be fluidly connected to the endoscope 100 through the connector portion 265 and the umbilical 260. A length of gas supply tubing 240c passes from one end positioned in an air gap 275 between the top 280 (e.g., bottle cap) of the reservoir 270 and the remaining water 285 in the reservoir to a detachable gas/lens wash connection 290 on the outside of the connector portion 265. The gas feed line 240b from the umbilical 260 branches in the connector portion 265 to fluidly communicate with the gas supply tubing 240c at the detachable gas/lens wash connection 290, as well as the air pump 215. A length of lens wash tubing 245c, with one end positioned at the bottom of the reservoir 270, may pass through the top 280 of the reservoir 270 to the same detachable connection 290 as the gas supply tubing 240c on the connector portion 265. In other embodiments, the connections may be separate and/or separated from each other. The connector portion 265 may also have a detachable irrigation connection 293 for irrigation supply tubing (not shown) running from a source of irrigation water (not shown) to the irrigation feed line 255b in the umbilical 260. In some embodiments, irrigation water is supplied via a pump (e.g., peristaltic pump) from a water source independent (not shown) from the water reservoir 270. In other embodiments, the irrigation supply tubing and lens wash tubing 245c may source water from the same reservoir. The connector portion 265 may also include a detachable suction connection 295 for suction feed line 250b and suction supply line 250a fluidly connecting a vacuum source (e.g., hospital house suction) (not shown) to the umbilical 260 and endoscope 100.

The gas feed line 240b and lens wash feed line 245b may be fluidly connected to the valve location 135 for the gas/water valve 140 and configured such that operation of the gas/water valve in the well controls supply of gas or lens wash to the distal tip 100c of the endoscope 100. The suction feed line 250b is fluidly connected to the valve location 135 for the suction valve 145 and configured such that operation of the suction valve 145 in the well controls suction applied to the working channel 235 of the endoscope 100.

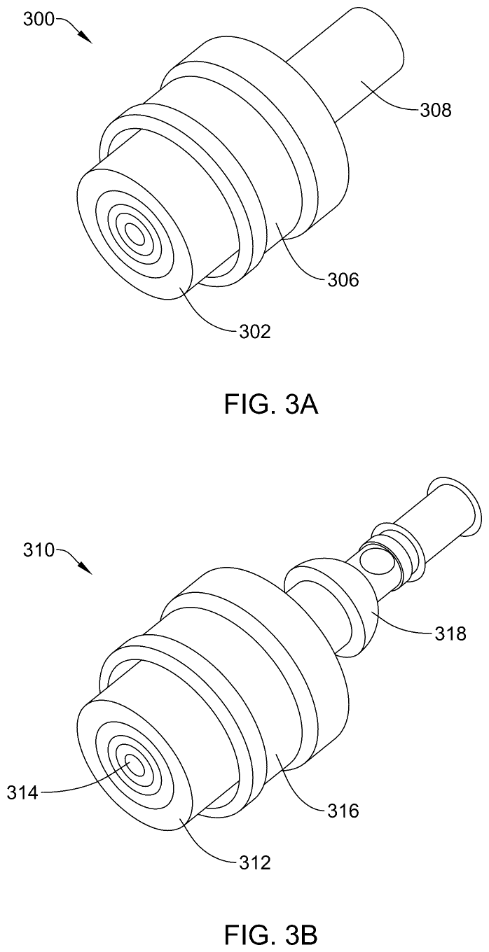

Examples of a removable suction valve 300 and a removable air/water valve 310 are illustrated in FIGS. 3A and 3B. For the removable suction valve 300, a valve cap 302 is connected to a valve body 306 by a resilient spring member. A shaft 308 is affixed to the valve cap 302 and moves with it. A user applies suction to the endoscope's working channel by pressing down on the cap 302, lowering the shaft 308 relative to the body 306 to close the valve 300. When the user ceases pressing down on the cap 302, the spring member returns the valve cap 302 and shaft 308 to the open position.

Similarly, the removable air/water valve 310 includes a valve cap 312 attached to a valve body 316 by a resilient spring member. In the center of the cap 312 is an air escape hole 314 connected to interior channels of the shaft 318. The gas feed line 240b vents through the escape hole 314 when the valve 310 is open. A user diverts air into the endoscope's working channel by covering the air escape hole 314 with a finger. To dispense water from the lens wash feed line 245b, the user presses down on the cap 312 with a finger, lowering the valve 310 into a closed position. When the user ceases pressing down on the cap 312, the spring member returns the valve 310 to the open position.

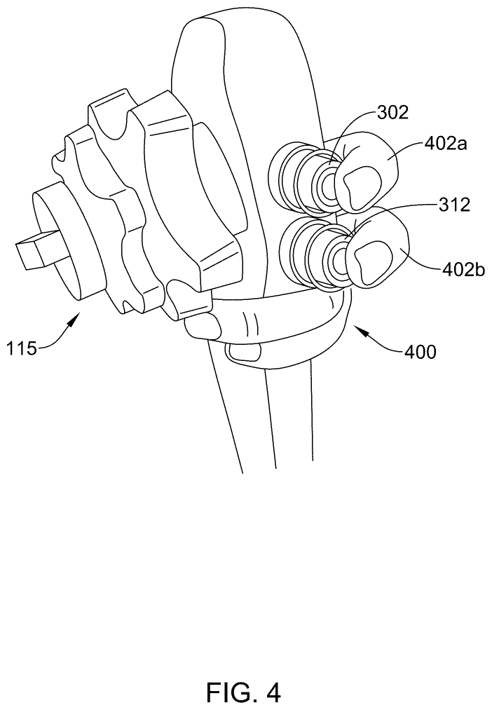

FIG. 4 shows an endoscopic handle 115 held by a user's hand 400. As shown, a first finger 402a is placed over the cap 302 of the suction valve 300, while a second finger 402b is placed over the cap 312 of the air/water valve 310. The valve locations 135 in the handle 115 are intended to allow this placement of fingers during operation.

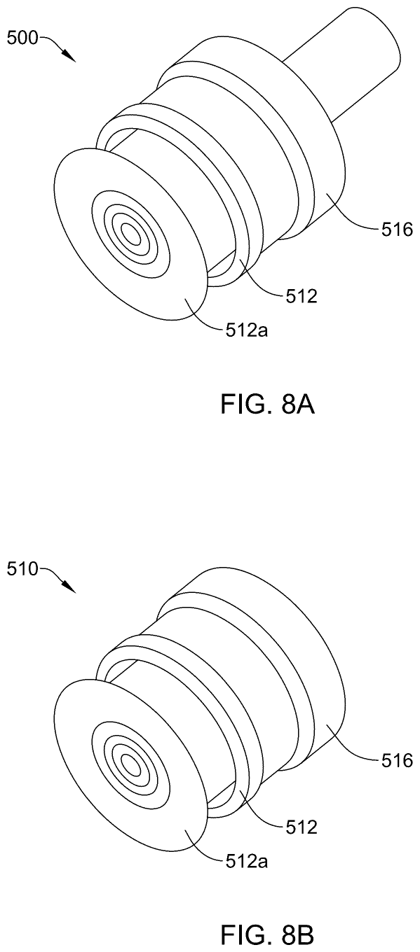

FIGS. 8A and 8B show a suction valve 500 and air/water valve 510 with valve caps having an expanded top surface area for ease of reach. The suction valve 500 has a cap 502 with an extended flange 502a that significantly extends the diameter of the cap 502. While the rest of the cap 502 is narrow enough to fit within the valve body 506, the flange 502a is wider than the opening provided in the body 506, and in some implementations may be wider than the body 506 altogether. The flange may be, in some implementations, at least 20% greater in diameter than the portion of the cap 502 that fits within the valve body 506. The top surface including the flange portion 502a may be made of the same or similar material as the remainder of the cap 502. The flange 502a may be formed monolithically as part of the cap 502 or may be formed separately and then attached to the cap 502.

Similarly, air/water valve 510 has a cap 512 with an extended flange 512a that significantly extends the diameter of the cap 512. While the rest of the cap 512 is narrow enough to fit within the valve body 516, the flange 512a is wider than the opening provided in the body 516, and in some implementations may be wider than the body 516 altogether. The flange may be, in some implementations, at least 20% greater in diameter than the portion of the cap 512 that fits within the valve body 516. The top surface including the flange portion 512a may be made of the same or similar material as the remainder of the cap 512. The flange 512a may be formed monolithically as part of the cap 512 or may be formed separately and then attached to the cap 512.

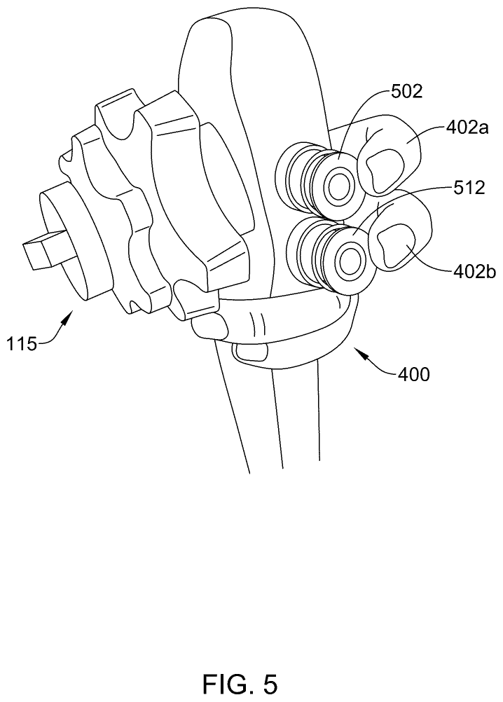

FIG. 5 shows an endoscopic handle 115 in which a suction valve 500 and air/water valve 510 have been inserted, each with an extended cap 502 and 512. Fingers 402a and 402b are each more easily able to be held by a user's hand 400. The first finger 402a is placed over the cap 502 of the suction valve 500, while a second finger 402b is placed over the cap 512 of the air/water valve 510.

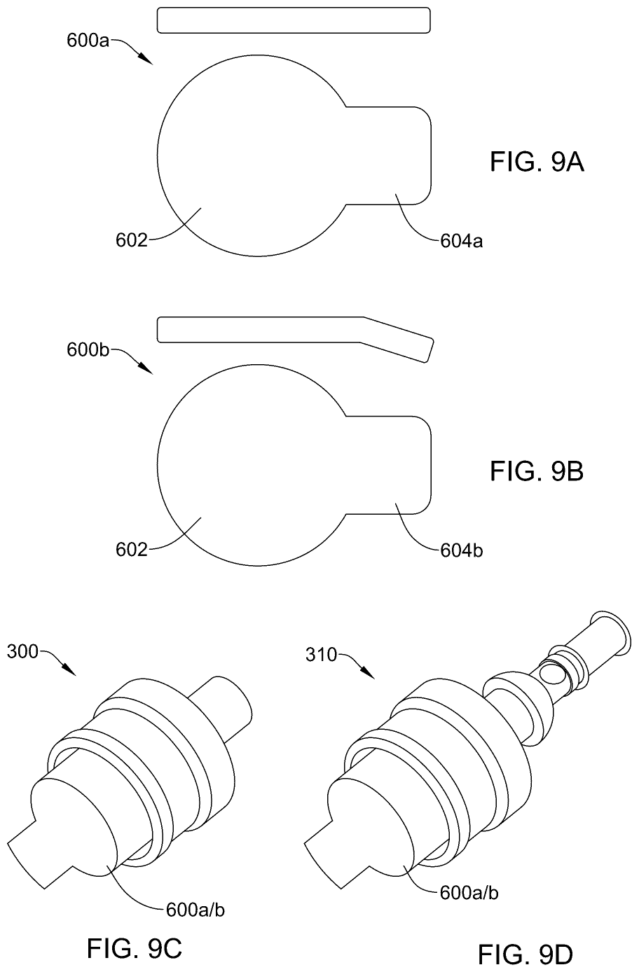

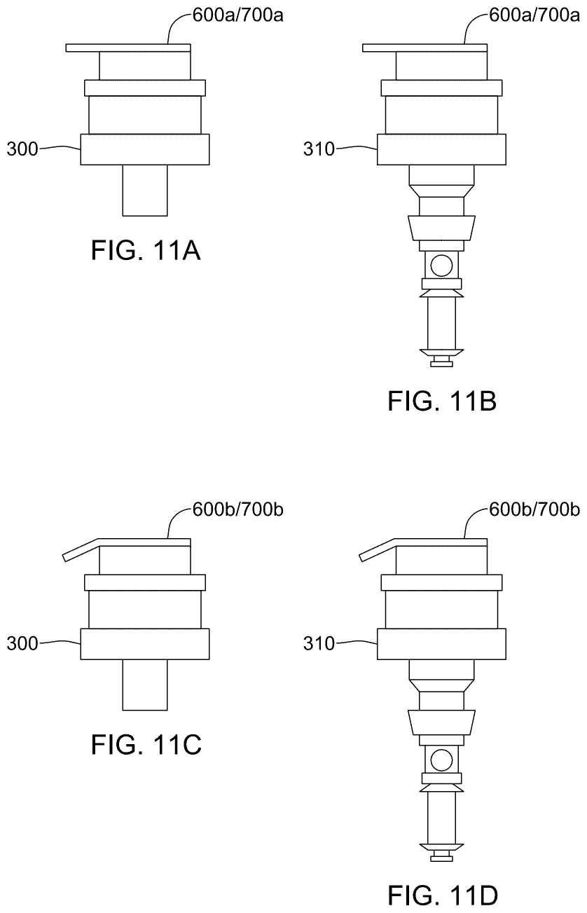

FIGS. 9A-11D show extensions that can be clipped on to typical endoscopic valves, such as suction valve 300 and air/water valve 310 as described above.

As shown in FIG. 9A, an extension 600a includes a surface portion 602 that can be removably attached to a valve 300 or 310. In some implementations, the surface portion 602 may be no wider than the surface of the valve cap to which it attached. A protrusion 604a extends from the surface portion 602 outward parallel to the surface. The protrusion 604a may be an integral part of the extension 600a, which may be molded or cut from a single piece of material (such as metal or thermoplastic). The surface portion 602 may be generally circular or otherwise rotationally symmetrical such that the operator may position the protrusion 604a at different angles to best accommodate the user's hand position.

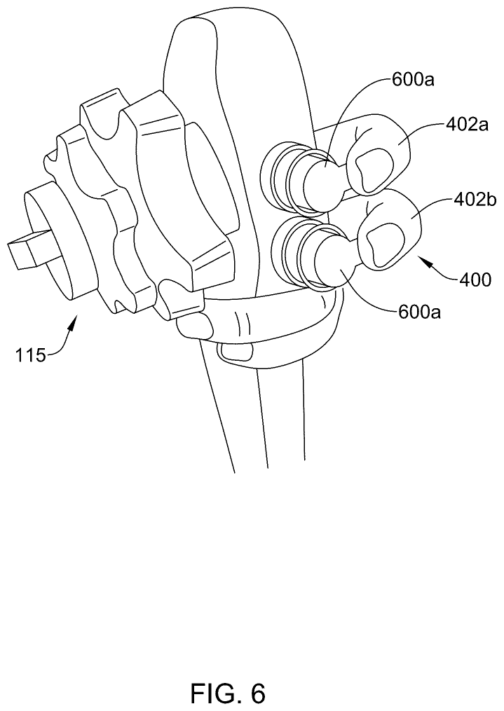

FIG. 9B shows a similar extension 600b including an identical surface portion 602. Here, the protrusion 604b extends at a downward angle, which may be selected based on ergonomic considerations. Again, the protrusion 604b may be integral with the rest of the extension 600b. FIGS. 6C and 6D show that the extension 600a or 600b attach to the suction valve 300 and the air/water valve 310 to extend the surface of these valves for interaction with a user.

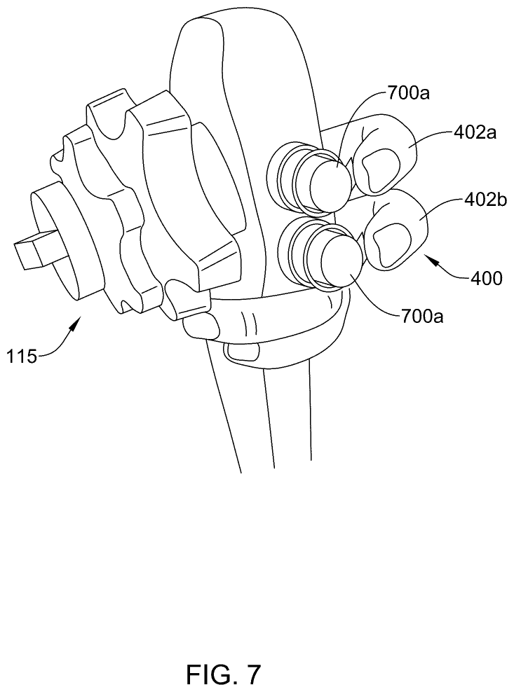

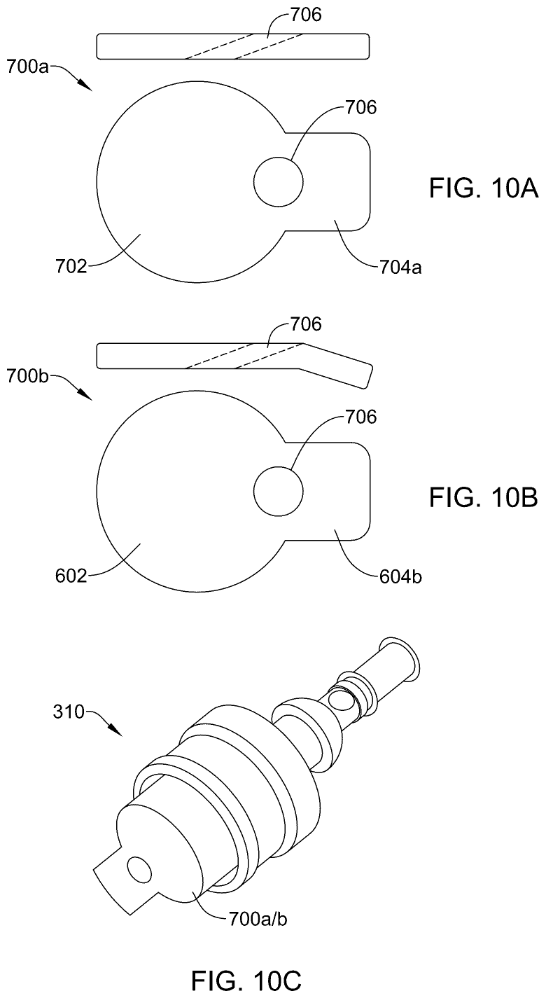

FIGS. 10A and 10B show extensions 700a and 700b which vary from the extensions 600a and 600b due to an air passage 706 extending therethrough. In some implementations, the air passage 706 matches the air escape hole 314 of the air/water valve 300. When attached to the air/water valve as illustrated in FIG. 10C, the extension 700a or 700b allows a user to operate the valve 300 from a more convenient position. A finger blocking the air passage 706 acts to block the air escape hole 314 as previously described. FIGS. 6 and 7 show how an operator's fingers can more easily reach these extensions.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A valve assembly for a medical device, comprising:
a valve body having an internal valve well;
a valve cap positioned within the internal valve well and configured to slide vertically within the internal valve well between an upper and a lower position;
a spring member positioned between the valve cap and the valve body such that, when the valve cap is pushed downward relative to the valve body, the spring member applies upward force against the valve cap; and
a valve shaft connected to the valve cap and configured to move with the valve cap between an upper and a lower position;
wherein the valve cap comprises a top flange having a width greater than a width of the internal valve well.

2. The valve assembly of claim 1, wherein the width of the top flange is more than 20% greater than the width of the internal valve well.

3. The valve assembly of claim 1, wherein the width of the top flange is greater than the width of the valve body.

4. The valve assembly of claim 1, wherein the valve cap has an air escape hole.

5. The valve assembly of claim 4, wherein the valve shaft has an air channel in fluid communication with the air escape hole.

6. The valve assembly of claim 1, wherein the valve assembly is an air/water supply valve.

7. The valve assembly of claim 1, wherein the valve assembly is a suction valve.

8. A valve assembly for a medical device, comprising:
a valve body having an internal valve well;
a valve cap positioned within the internal valve well and configured to slide vertically within the internal valve well between an upper and a lower position;
a spring member positioned between the valve cap and the valve body such that, when the valve cap is pushed downward relative to the valve body, the spring member applies upward force against the valve cap;
a valve shaft connected to the valve cap and configured to move with the valve cap between an upper and a lower position; and
a removable valve cap extension attached to the valve cap, the removable valve cap extension having a protrusion extending past an edge of a top surface of the valve cap.

9. The valve assembly of claim 8, wherein the removable valve cap extension comprises a surface portion configured to attach to the top surface of the valve cap.

10. The valve assembly of claim 9, wherein the surface portion rotationally symmetrical such that the protrusion can be positioned to extend from the top surface of valve cap at different angles.

11. The valve assembly of claim 8, wherein the protrusion of the removable cap extension extends parallel to the top surface of the valve cap.

12. The valve assembly of claim 8, wherein the protrusion of the removable cap extension extends at a downward angle from the surface of the valve cap.

13. The valve assembly of claim 8, wherein the valve cap comprises an air escape hole, and wherein the removable cap extension comprises an air passage in fluid communication with the air escape hole.

14. The valve assembly of claim 13, wherein the air passage of the removable cap extension runs at an angle from a bottom of the surface portion towards a top of the protrusion.

15. An endoscopic surgical device, comprising:
an operating handle having first and second valve locations;
a source of suction in fluid communication with the first valve location;
a first valve assembly installed in the first valve location;
a gas supply in fluid communication with the second valve location;
a water supply in fluid communication with the second valve location; and
a second valve assembly installed in the second valve location;
wherein the first valve assembly and the second valve assembly each comprises:
a valve body having an internal valve well,
a valve cap positioned within the internal valve well and configured to slide vertically within the internal valve well between an upper and a lower position,
a spring member positioned between the valve cap and the valve body such that, when the valve cap is pushed downward relative to the valve body, the spring member applies upward force against the valve cap,
a valve shaft connected to the valve cap and configured to move with the valve cap between an upper and a lower position, and
an upper surface at the top of the valve assembly having a width significantly greater than a width of the internal valve well.

16. The endoscopic surgical device of claim 15, wherein the upper surface at the top of the valve assembly is an upper surface of a top flange of the valve cap.

17. The endoscopic surgical device of claim 16,
wherein the valve cap of the valve assembly has an air escape hole; and
wherein the valve shaft of the valve assembly has an air channel in fluid communication with the air escape hole.

18. The endoscopic surgical device of claim 15, the valve shaft further comprising a removable valve cap extension having a protrusion extending past an edge of a top surface of the valve cap;
wherein the upper surface at the top of the valve assembly is the upper surface of the removable valve cap extension including the upper surface of the protrusion.

19. The endoscopic surgical device of claim 18, wherein the valve cap of the valve assembly comprises an air escape hole, and wherein the removable cap extension of the valve assembly comprises an air passage in fluid communication with the air escape hole.

20. The endoscopic surgical device of claim 19, wherein the air passage of the removable cap extension runs at an angle from a bottom of the surface portion towards a top of the protrusion.
